(19) United States
(12) Patent Application Publication
(10) Pub. No.: US 2008/0038252 A1
(43) Pub. Date: Feb. 14, 2008

(54) Histamine-Containing Composition for the Treatment of Allergic Diseases

(76) Inventors: Sook-yeong Jeon, Seoul (KR); Dong-ho Nahm, Seoul (KR)

Correspondence Address: THE RAFFERTY PATENT LAW FIRM, 5641 BURKE CENTRE PKWY, SUITE 100, BURKE, VA, 22015-2259, US

(21) Appl. No.: 11/908,806

(22) PCT Filed: Mar. 17, 2006
(86) PCT No.: PCT/KR06/00996

§ 371 Date: Sep. 19, 2007

(30) Foreign Application Priority Data
Mar. 18, 2005 (KR) 10-2005-0022558

(51) Int. Cl. A61K 39/395; A61P 37/08

(52) U.S. Cl. 424/130.1

(57) ABSTRACT

The present invention provides a pharmaceutical composition comprising histamine, immunoglobulin and allergen as active ingredients. Also, the present invention provides a kit for treating allergic diseases comprising the above active ingredients. The present invention also provides use of a composition comprising histamine, immunoglobulin and allergen as active ingredients for the manufacture of medicament for treating allergic diseases. Further, the present invention provides a method of treating allergic diseases which comprises administrating the pharmaceutical composition comprising a therapeutically effective amount of histamine, immunoglobulin and allergen to a mammal. Therefore, the allergic diseases can be significantly improved in the patients with refractory allergic diseases who could not be sufficiently improved by treatment with standard drug therapy or allergen-immunotherapy if the pharmaceutical composition, its use for treating allergic diseases, or the treating method using the above compositions of the present invention was applied.

### TECHNICAL FIELD

The present invention relates to pharmaceutical compositions for treatment of allergic diseases, a kit for treating allergic diseases, the use of the above said compositions for the manufacture of medicament for treating allergic diseases, and a method of treating allergic diseases.

### BACKGROUND ART

Hypersensitive immune response to specific antigenic materials (allergic reaction) has been regarded as a pathogenetic mechanism responsible for the development of allergic diseases including atopic dermatitis, allergic rhinitis, allergic conjunctivitis, urticaria, and allergic asthma (Bierman C W, et al. (eds.) Allergy, asthma, and immunology from infancy to adulthood. page xvii, Saunders, Pa., 1996). Generally, an antigenic material that can induce allergic reaction or IgE antibody-mediated immediate hypersensitivity reaction is defined as an allergen in the fields of the allergy. Recently the prevalences of allergic diseases are rapidly increasing in the world due to changes in environmental factors. However, the fundamental treatments of allergic diseases are difficult because the pathogenetic mechanisms of allergic diseases are not completely defined yet.

Allergic reaction and allergic diseases are currently known to be developed as following steps.

(1) A subject with specific genetic predisposition is exposed to the environmental agents that can induce a hypersensitive immune response (=allergen such as house dust mites, pollens, animal dander, molds) and produce antibodies to these allergens and allergen-specific T lymphocytes. This process is expressed as “being sensitized to allergen.”

(2) The above subject sensitized to specific allergen is exposed to the same allergen through the airway, eye, gastrointestinal mucosa, or skin. When the allergen binds to the allergen-specific IgE or IgG antibodies on the surface of IgE or IgG receptor bearing cells including mast cell or the allergen react with allergen-specific T cells, various chemical mediators including histamine are released from the mast cells or T cells.

(3) The above chemical mediators released from the IgE and IgG receptor bearing cells or T cells induce a chronic inflammation of tissues including airway, eye, and skin and produce the hypersensitive state of the above tissues, and finally result in various clinical symptoms associated with allergic diseases including constriction of airway, respiratory difficulties, itching of nasal mucosa and eye, sneezing, edema of airway, urticaria, itching of skin, eczema, scaling of skin, and thickening of skin in above the subject.

The above allergic reaction is a kind of immunological hypersensitivity reaction and classified into type I, type II, type III, and type IV hypersensitivity reactions (Gell and Coombs' classification of hypersensitivity reaction). Generally, typical allergic reaction indicates type I hypersensitivity reaction mediated by IgE antibodies, but type II, type III, and type IV hypersensitivity reactions also have been suggested to be involved in the pathogenesis of allergic diseases. Accordingly, allergic reaction in wide meaning indicates all kinds of hypersensitive immunological reaction and resulting phenomenon that can be harmful to the host (Bierman C W, et al. (eds.) Allergy, asthma, and immunology from infancy to adulthood. page xvii, Saunders, Pa., 1996).

Besides, the above allergic diseases including atopic dermatitis, allergic asthma, allergic conjunctivitis, allergic rhinitis, and urticaria have been shown to be developed by similar pathogenetic mechanisms although their clinical characteristics are differently expressed. Accordingly there are many patients who are suffering from more than 2 kinds of above allergic diseases at the same time and even some patients have all the 5 kinds of above allergic diseases at the same time.

Currently, three kinds of treatment methods are used and these methods include avoidance of allergen, desensitization, and the use of drugs.

Of these, avoidance of the allergen is a method to avoid exposure to causative allergens identified to be relevant allergen to the patient. Causative allergen to the animal or human subject can be identified by allergy skin test or by in-vitro tests for specific IgE antibodies to allergens in serum samples (Board of Directors. Allergen skin testing. J Allergy Clin Immunol 92:653-7, 1993; Bierman C W, et al. (eds.) Allergy, asthma, and immunology from infancy to adulthood. p 144-156, Saunders, Pa., 1996). Avoidance of allergens is theoretically an ideal method. However, decreasing the exposures to common allergens including house dust mite and pollens enough to markedly improve the clinical symptoms in patients with allergic diseases is frequently difficult yet.

Current pharmaceutical treatment is directed to improve the symptoms of allergic disease produced by the allergic reaction to causative allergens and suppress the resulting inflammations of various tissues and suppress the resulting physical responses to chemical mediators released during the allergic reaction. However, current pharmacological therapy can improve the clinical symptoms and physiological functions only during the continuous administration of medication and is not yet proven to fundamentally improve the allergic diseases. Currently, systemic administrations of drugs for the treatment of allergic diseases including corticosteroids, leukotriene antagonists, antihistamines, and anti-IgE antibody therapies are known to simultaneously improve the most of clinical symptoms related to different allergic diseases, even if the patients have more than 2 kinds of allergic diseases.

The third method of treatment for allergic diseases is an allergen-immunotherapy (also called as a desensitization therapy). Allergen-immunotherapy is a treatment that causative allergen is administered to the patients with allergic diseases from the small dose to increasing dose at regular intervals through the subcutaneous, sublingual, or oral routes and resulted in a decreased hypersensitivity reaction to the causative allergens and eventually fundamentally improve the allergic diseases.

Usually, the subcutaneous injection of allergen is known to be the preferred form of allergen-immunotherapy. After the initial development of allergen-immunotherapy by Noon at 1911 (Lancet 1911;1:1572-3), the allergen-immunotherapy still remains as a very useful treatment method for allergic diseases (Bousquet J. et al.; J Allergy Clin Immunol 1998;102:558-62). Especially, allergen-immunotherapy is known to be effective for the allergic asthma, allergic rhinitis, allergic conjunctivitis, and bee-venom allergy (Bousquet J. et al.; J Allergy Clin Immunol 1998;102:558-62).

Interestingly, the mechanism of allergen-immunotherapy is not completely defined yet and some patients who received allergen-immunotherapy are not sufficiently improved to the state without needs for additional pharmaceutical treatment. Especially, the allergen-immunotherapy is effective only for a small subgroup of patients with atopic dermatitis and currently is not recommended as a standard therapy for atopic dermatitis by the evidence-based guidelines or by the majority of the experts (Hanifin J M, et al. Guidelines of care for atopic dermatitis. J Am Acad Dermatol 2004;50:391-404.). When the children having both respiratory allergic diseases (allergic asthma and/or allergic rhinitis) and atopic dermatitis simultaneously were treated with allergen-immunotherapy for 3 years, respiratory allergic diseases were improved in 70% of patients but the atopic dermatitis was not improved in 80% of patients in the previous report by Wuthrich (Wuthrich B. Ann Allergy 1999;83:464-70). This result suggests that current traditional allergen-immunotherapy is not significantly effective for the treatment of atopic dermatitis. A significant portion of patients with atopic dermatitis is not effectively controlled by the current standard treatments including topical moisturizing agents, topical corticosteroids, and topical immunosuppressants. Although a systemic administration of corticosteroids or immunosuppressants can improve the clinical symptoms in patients with refractory atopic dermatitis, these treatments usually could not be continued for long terms due to a increased possibility of systemic side effects (Ellis C, et al. Bri J Dermatol 2003(Suppl. 63):3-10).

The effectiveness of allergen-immunotherapy is known to be dose-dependent. However high dose of allergen-immunotherapy is associated with increased risk for local and systemic side effects. Localized side effects of allergen-immunotherapy include swelling, pain, and eruption on the injection site and systemic side effects of allergen-immunotherapy include generalized urticaria, respiratory difficulty, dizziness, and shock. Especially, there have been reports about patients who were died due to systemic allergic reactions after allergen-immunotherapy (Lockey R F, et al. Ann Allergy Asthma Immunol 2001;8747-55). The frequencies of systemic side effects during allergen-immunotherapy are various from 0.8% to 46.7% according to the reporters in the previous reports. In a previous report of 386 patients with allergic diseases who received 22,722 injections for allergen-immunotherapy, the systemic side effects were experienced in 6.22% of patients and the mean frequency of systenic side effects was 0.12% per one injection (Ohashi Y, et al. Acta Otolaryngol 1998;538:113-7). In the above report, the incidence of systemic side effects in patients with atopic dermatitis was 43.75% which was significantly higher incidence compared to the 2.82% in patients without atopic dermatitis. The practice rate of allergen-immunotherapy in the management of patients with allergic diseases is known to be widely different according to the countries due to the safety issues of allergen-immunotherapy.

Currently, there are various trials to maximize the therapeutic effects and minimize the side effects of allergen-immunotherapy (Casale T B, et al. J Allergy Clin Immunol 2006;117.134-40).

As a trial to increase the effect and decrease the side effect of allergen-immunotherapy, Saint-Remy, et al. developed an immunotherapy method to intradermally inject the immune complex made of allergens and allergen-specific antibodies isolated from a patient with allergic diseases into the same patient from small dose to the sequentially increasing doses (Saint-Remy J M, et al. Clin Exp Allergy 1994;24: 1091-3). In the above treatment method, the allergen-specific antibodies were purified from the plasma sample of the same patient with allergic diseases by affinity chromatography method using allergen coupled agarose-beads. This treatment method has been reported to be clinically effective and relatively safe for the treatment of the patients with atopic dermatitis and allergic asthma who are sensitized to house dust mite (Saint-Remy J M, et al. Clin Exp Allergy 1994;24:1091-3). However, the above allergen-immunotherapy using immune complex made of allergen and allergen-specific antibodies is difficult to be widely used because the production of the above therapeutic composition is very complex and expensive due to the need of isolating allergen-specific antibodies from individual patients and carries various risks including the contamination of infectious agents during the production process. Accordingly, clinically beneficial effects of the allergen-immunotherapy using immune complex made of allergen and allergen-specific antibodies in the treatment of allergic diseases have not been reproduced by other researchers beside the Saint-Remy group that reported the clinical effectiveness for the first time. So, the above allergen-immunotherapy using immune complex made of allergen and allergen-specific antibodies is not regarded as a standard therapy in the international guidelines of treatment for atopic dermatitis due to the lack of sufficient evidences supporting the clinical effects and safety of the above treatment method (Hanifin J M, et al. J Am Acad Dermatol 2004;50:391-404).

In 1951, Parrot and Laborde developed a new treatment method administering a complex of histamine and human serum gammaglobulin to restore the ability to block the biologic activity of histamine (histaminopexy) that is decreased in the sera of patients with allergic diseases (J Physiol 1951;40:885-9). A complex of histamine/human gammaglobulin has been widely used in many countries including Japan ad Europe for the treatment of allergic diseases such as allergic rhinitis, bronchial asthma, atopic dermatitis, and chronic urticaria (Yoshii H, et al. J Allergy Clin Immunol 1997;100:809-16). The treatment method using histamine-immunoglobulin complex is sometimes named as ‘nonspecific immunotherapy.’ Injection of histamine-immunoglobulin complex to allergy animal model induced reductions of allergic inflammatory reaction and also showed an immunomodulating effects inducing reductions of the serum levels of TNF-alpha, IL-4 and allergen-specific IgE antibodies (Ayoub M, et al. Int Immunopharmacol 2003;3:523-539). The anti-inflammatory effect of a complex of histamine/gammaglobulin was not produced when the same amount of histamine or gammaglobulin alone was administered to the allergy animal model (Yoshii H, et al. J Allergy Clin Immunol 1997;100:809-16). Accordingly, some interaction between these two materials was supposed to have an important role in the pharmacological effects of this histamine/gammaglobulin complex on the treatment of allergic diseases. However, the ‘nonspecific immunotherapy’ using histamine-immunoglobulin complex is not recommended as a standard pharmacological treatment in the international guidelines for treatment of allergic diseases because the clinical effectiveness of histamine-immunoglobulin complex is not significantly better than the effectiveness of other current standard pharmacological treatments (Hanifin J M, et al. J Am Acad Dermatol 2004;50:391-404).

The ‘allergen-immunotherapy’ and ‘nonspecific immunotherapy’ using histamine-immunoglobulin complex has been respectively used as an independent therapy or a concomitant therapy administered with other pharmacological therapies until now and each type of the immunotherapy has some weakness as described above. However, a combination of ‘allergen-immunotherapy’ using allergen and ‘nonspecific immunotherapy’ using histamine-immunoglobulin complex by administrating the mixture of the above two treatment compositions simultaneously for the treatment of allergic diseases has never been tried yet.

### DISCLOSURE OF INVENTION

Technical Problem

The present inventors made great efforts to develop a more effective pharmaceutical composition and a new treatment method for the improvement of patients with severe allergic diseases that is not effectively controlled by current standard pharmacological therapies. As one of these efforts, the present inventors judged that a combination of ‘allergen-immunotherapy’ using allergen and ‘nonspecific immunotherapy’ using histamine-immunoglobulin complex might be more effective compared than the each of the above two treatment methods alone and developed a new pharmaceutical composition and a new treatment method of the present invention.

The present inventors at the first time subcutaneously injected a histamine-immunoglobulin complex to the other arm not receiving subcutaneous injections of allergen as a maintenance stage of allergen-immunotherapy in the patients with allergic diseases who were not effectively improved by allergen-immunotherapy alone. However, the present inventors mixed the allergen solution from the vials for allergen-immunotherapy with lyophilized powder in a vial containing human immunoglobulin and histamine dichloride together and subcutaneously injected the above dissolved mixture at once into single arm to reduce the pains from two separate injections of two different compositions into both arm to single injection. Unexpectedly and surprisingly, the inventors found that monthly injections of mixture of allergen solution for allergen-immunotherapy with histamine-immunoglobulin complex resulted in remarkable improvements of allergic disease compared to the results from the allergen-immunotherapy or histamine-immunoglobulin complex treatment alone.

Technical Solution

The present invention provides a pharmaceutical composition comprising histamine, immunoglobulin and allergen as active ingredients.

The present invention also provides a pharmaceutical composition for treating allergic diseases comprising histamine, immunoglobulin and allergen as active ingredients.

Also, the present invention provides a kit for treating allergic diseases comprising: a first container containing histamine; a second container containing immunoglobulin; and a third container containing allergen.

The present invention also provides a kit for treating allergic diseases comprising: a first container containing one or more ingredients selected from a group consisting of histamine, immunoglobulin and allergen; and a second container containing other ingredient(s).

The present invention provides the use of a composition comprising histamine, immunoglobulin and allergen as active ingredients for the manufacture of medicament for treating allergic diseases.

Further, the present invention provides a method of treating allergic diseases which comprises administrating a pharmaceutical composition comprising a therapeutically effective amount of histamine, immunoglobulin and allergen to a mammal.

In an embodiment of the present invention, the allergic diseases can be atopic dermatitis, allergic rhinitis, allergic conjunctivitis, urticaria or allergic asthma.

Also, in an embodiment of the present invention, the allergen can be house dust mite, pollen, animal dander or fungus.

In an embodiment of the present invention, the active ingredients can be present in the form of dry powder. They can be dissolved in buffer for injection, and then administrated to a mammal.

In an embodiment of the present invention, the kit for treating allergic diseases can further comprise a container containing buffer for injection.

The present invention will be described more specifically as belows.

Unless indicated differently, the terms used in the present invention will be regarded to be used as same meaning over the present specification.

The present invention provides a pharmaceutical composition comprising histamine, immunoglobulin and allergen as active ingredients.

The pharmaceutical composition comprising histamine, immunoglobulin and allergen as active ingredients can be used for treating allergic diseases. Thus, the present invention provides a pharmaceutical composition for treating allergic diseases comprising histamine, immunoglobulin and allergen as active ingredients.

The term “composition” as used hereinbefore or hereinafter is regarded as including any product formed by combination of specific ingredients directly or indirectly as well as a product containing the specific ingredients.

Each of ingredients used in the present composition can be present separately or complexly in the present composition, in the injectable formulation in which the present composition is dissolved, or in living bodies. For instance, histamine and immunoglobulin can form histamine-immunoglobulin complex which are bonded covalently or non-covalently. Also, histamine, immunoglobulin or histamine-immunoglobulin complex can form complex with allergen in the present composition, in the injectable formulation in which the present composition is dissolved, or in living bodies.

The present composition includes a composition in which one of active ingredients is pharmaceutically or physiologically acceptable salt, a composition in which all of active ingredients are pharmaceutically or physiologically acceptable salts, a composition in which one of active ingredients is pharmaceutically or physiologically acceptable salt and the other ingredient(s) is free base form or a composition in which the complex of one or more ingredients is pharmaceutically or physiologically acceptable salt.

The salts of the active ingredients or the complex of one or more ingredients in the present composition are meant to comprise all forms of pharmaceutically or physiologically acceptable salts. The pharmaceutically or physiologically acceptable salts of the active ingredients or the complex of one or more ingredients in the present composition includes water-soluble, oil-soluble or insoluble salt forms. For example, they include the conventional non-toxic salts or the quaternary ammonium salts which are formed, e.g., from organic or inorganic acids or bases. Examples of such acid addition salts include acetate, adipate, alginate, aspartate, benzoate, benzene-sulfonate, bisulfate, butyrate, citrate, camphorate, camphorsulfonate, cyclopentane-propionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, phosphate, propionate, succinate, sulphate, tartrate, thiocyanate, tosylate, undecanoate, etc. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such a sarginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl, and diamyl sulfates; long chain halides such as decyl, lauryl, myristyl and stearyl chlorides, bromides and iodides; aralkyl halides like benzyl and phenethyl-bromides and others. Other pharmaceutically or physiologically acceptable salts include the sulfate salt ethanolate and sulfate salts.

An active ingredient, “histamine” in the composition of the present invention is a compound of formula C₅H₉N₃, which is broadly present within living bodes. It is formed from decarboxylation of histidine in protein by putrefactive bacterium or enteric bacterium. It is regarded that histamine is present in tissues as inactive form which is bonded with tissue protein, but when allergic reaction or anaphylaxis is developed, the inactive histamine become to be active form by certain action, thereby the activated histamine acts to organs or tissues. Histamine used in the present composition can be chemically prepared by well known methods in the art, or can be a selling good obtained from the art.

Another active ingredient, “immunoglobulin” in the composition of the present invention is defined as protein having important role in immunity and acting as antibody, among serum components. Basic structure of irrmunoglobulins consists of a pair of L chain (light chain) having molecular weight of about 23,000 and a pair of H chain (heavy chain) having molecular weight of about 50,000 to 70,000, wherein the L chain and H-chain are linked to each other by S—S bond. Immunoglobulins are classified to IgG, IgA, IgM, IgD, IgE according to the kinds of H chain, i.e., γ, α, μ, δ, ε. The immunoglobulin used in the present composition can be IgG, IgA, IgM, IgD, IgE or their mixture thereof, their fragments having biologically equal activity or the mixture of the fragments. Also, the immunoglobulin used in the present composition can comprise specific or non-specific immunoglobulin to allergen. The immunoglobulin used in the present composition can be isolated from plasma of human or animal. Generally, the immunoglobulin of the present composition can be formulated by plasma fractionation, or prepared by well-known genetic engineering technique.

The other active ingredient “allergen” in the present composition generally indicates a specific antigen which can induce allergic reaction or IgE-antibody mediated hyper-sensitivity reaction. Allergen can be common materials such as mite, pollen, animal dander, fungus, food, synthetic fiber, accessory, drug, cosmetics, etc. Most of common materials contacted by eating, touching, breathing can be an allergen. Allergen can be broadly classified to inhalant allergen, food allergen, drug allergen, or contact allergen. Inhalant allergen means materials which were inhaled into living body during respiration. It can include pollen, house dust mite, animal (including dog, cat, ect.) dander, fungus, adhesive, paints, etc. Food allergen means materials which can induce hypersensitivity reaction or allergy reaction among eatable materials. It can include egg, milk, milk products, meat, bean, buckwheat, shrimp, crab, peach, processed food, etc. Drug allergen means materials which can induce hypersensitivity reaction or allergic reaction by getting into living body by injection or as an oral medication. It can include antibiotics, analgesics, hormone drugs, etc. Contact allergen means materials which can induce hypersensitivity reaction or allergy reaction by contacting to skin. It can include cosmetics, dye, clothing, detergent, rubber, metal, chemical materials, etc.

Preferably, the allergens used in the present composition may be, but are not limited to, pollen, house dust mite, animal dander, fungus or their mixtures which can frequently induce allergic reaction in many people. Preferably, patients with allergic diseases can be divided into several groups according to the kind of allergen (sensitized) showing allergic reaction in each of patients, and then the allergen used in the present composition can be consisted of single allergen component or mixture of several allergen components suitable for the treatment of the allergic diseases in such patient groups. The causal allergen can be confirmed by serum allergen-specific IgE antibody test or skin test administrating allergen into skin according to known method to observe skin flare, wheal or edema (Board of Directors. Allergen skin testing. J Allergy Clin Immunol 92:653-7, 1993; Bierman C W, et al. (eds.) Allergy, asthma, and immunology from infancy to adulthood. p 144-156, Saunders, Pa., 1996).

To prepare the pharmaceutical compositions of this invention, the active ingredients can be combined in intimate admixture with a pharmaceutically acceptable carrier, which carrier may take a wide variety of forms depending on the form of preparation desired for administration. These pharmaceutical compositions are desirably in unitary dosage form and can take dilutable form to control dosage depending on doctors's judgment.

The composition of the present invention is to use for subcutaneous injection. In embodiments of the present invention, however, the composition can be administered intravenously, intraarterially, intramuscularly, intraperitoneally, intrasternally, percutaneously, intranasally, rectally, orally, intraocularly, intraderially, locally, or by inhalation, according to ordinary methods.

Buffer for injection and other additive components to prepare the present composition to an injection formulation are well-known in the art. The injection formulation of the present composition can comprise additive components such as solubilizers, pH adjusting agents, suspending agents, etc., besides the buffer for injection. As the buffer for injection, physiological saline, etc. can be used.

The present composition can be used for treating allergic diseases. The examples of treatable allergic diseases can include, but are not limited to, atopic dermatitis, allergic rhinitis, allergic conjunctivitis, urticaria or allergic asthma.

Also, the present invention provides a kit for treating allergic diseases comprising containers which contain active ingredient(s) in the pharmaceutical composition separately or together.

In an embodiment of the present invention, this invention provides a kit for treating allergic diseases comprising: a first container containing histamine; a second container containing immunoglobulin; and a third container containing allergen.

Otherwise, the present invention provides a kit for treating allergic diseases comprising: a first container containing one or more ingredients selected from a group consisting of histamine, immunoglobulin and allergen; and a second container containing other ingredient(s).

The kit for treating allergic diseases of the present invention can further comprise a container containing buffer for injection.

The allergen contained in the kit can be house dust mite, pollen, animal dander or fungus, and the active ingredients in the kit can be present in the form of dry powder. Also, the allergic diseases can be atopic dermatitis, allergic rhinitis, allergic conjunctivitis, urticaria or allergic asthma.

The container used for the kit can be glass or plastic container which can comprise the active ingredient(s) and can be sealed hermetically. It is sufficient the container can comprise the active ingredient(s), but the container is not required to have specific form.

The present invention provides use of a composition comprising histamine, immunoglobulin and allergen as active ingredients for the manufacture of medicament for treating allergic diseases. The pharmaceutical composition comprising histamine, immunoglobulin and allergen as active ingredients can be used for the manufacture of medicament for treating allergic diseases.

Also, the present invention provides a method of treating allergic diseases which comprises administrating a pharmaceutical composition comprising a therapeutically effective amount of histamine, immunoglobulin and allergen to a mammal.

The term “mammal” as used hereinbefore or hereinafter means mammal as a subject for treatment, observation or examination, preferably, human.

The term “therapeutically effective amount” used hereinbefore or hereinafter means that amount of active ingredient or pharmaceutical composition that elicits the biological or medicinal response in a tissue, system animal or human by a researcher, veterinarian, medical doctor or other clinician, which can induce alleviation of the symptoms of the disease or disorder being treated.

The method for treating allergic diseases in the present invention can be performed by using the abovely-described pharmaceutical composition.

The dosage of the present pharmaceutical composition can be decided by considering the dosage of allergen and histamine-immunoglobulin complex used in allergen-immunotherapy and non-specific immunotherapy using histamine-immunoglobulin complex. Though the dosage of general pharmaceutical composition can be decided depending on severity of the clinical symptoms, age, weight of the patient, etc., the dosage of the present composition should be decided by considering patient's sensitivity for the allergen causing the allergic diseases and/or patient's sensitivity for histamine or immunoglobulin, as well as the above condition.

When the present pharmaceutical composition comprising histamine, immunoglobulin and allergen is administrated at once, a dosage of histamine can be 0.05 to 2.5 μg, preferably 0.1 to 1.0 μg, more preferably 0.15 to 0.45 μg. Also, when the pharmaceutical composition is administrated at once, a dosage of immunoglobulin can be 0.05 to 50 mg, preferably 12 to 36 mg, and a dosage of allergen can be 1 to 1000 μ/ml, preferably 50 to 100 μg/ml as protein amount in buffer for injection. Preferably, histamine, immunoglobulin and allergen can be combined and then dissolved in buffer for injection of 0.5 to 2 ml at once.

Preferably, histamine, immunoglobulin, allergen and other additive components are provided in the forms of sealed separately, and then dissolved to use before administration according to the doctor's decision for the dosage depending on condition of the patient.

The dosage of the above active ingredients at the time of treating allergic diseases is not fixed, and can be increased gradually considering the sensitivity of the patients to the first administration dose. Also, at time of administration of the pharmaceutical composition the dosage of the histamine and immunoglobulin can be maintained constantly, and according to increasing the number of administration the concentration of allergen can be increased to strengthen immunity to the allergen in the patients. A dosage of the pharmaceutical composition can be controlled by the doctor's decision with wide experience considering the patient's condition according to the administration of the present composition.

It is evident for a skilled artisan that the therapeutically effective amount of the active ingredients and the pharmaceutical composition comprising the active ingredients of the present invention and the number of their administration will be varied depending on desirable effect. Therefore, the most suitable dosage to be administrated can be decided easily, and it can be varied depending on certain active ingredient to be used, mode of administration, effect of formulation and development of the diseases condition. Also, it will be needed to control the dosage of administration to adjust treatment level appropriately depending on patients individual factors including age, body weight, diet, and timing of administration, etc.

The advantages and features of the present invention and the method of revealing them will be explicit from the following examples described in detail. However, it is to be distinctly understood that the present invention is not limited thereto but may be otherwise variously embodied and practiced. It is obvious that the following examples are to complete the disclosure of the invention and to indicate the scope of the present invention to a skilled artisan completely, and the present invention will be defined only by the scope of the claims.

### MODE FOR THE INVENTION

### EXAMPLES

### Example of Formulation 1

Human immunoglobulin 12 mg

Histamine dichloride 0.15 μg

Sodium chloride 4 mg

Amino acetic acid 45 mg

D-manitol 4 mg

Sodium hydroxide an appropriate amount

Aluminum hydroxide 0.001-2 mg

Allergen 40-60 μg (as a protein amount)

Solution for injection 0.8-2 ml (supplied as another vial from the above ingredients)

### Example of Formulation 2

Formulations of injection solution for allergen-immunotherapy and histamine-immunoglobulin complex used in the Examples of the present invention and methods for their administration.

### Example of Formulation 2-1: Injection Solution for Allergen-Immuotherapy

An injection solution for house dust mite allergen-immunotherapy (Novo-Helisen Depot™; Allergopharma Joachim Ganzer K G, Reinbeck, Germany) containing house dust mite extracts (50%: 50% mixture of Dermatophagoides farinae and Dermatophagoides pteronyssinus extracts) adsorbed to aluminum hydroxide was administered as recommended by manufacturer. The above injection solution of allergen-immunotherapy (Novo-Helisen Depot™; Allergopharma Joachim Ganzer K G, Reinbeck, Germany) used in the maintenance therapy schedules contained 60-80 μg/ml of protein quantified by Bradford's method and also contained aluminum hydroxide, 0.4% phenol, and saline solution; the concentration of house dust mite extracts was indicated as 5000 therapeutic unit (TU)/ml in the information of the product provided by the manufacturer. The above treatment kit is composed of 3 vials (No. 1, 2, 3) of allergen solutions for injection in increasing concentrations. The concentration of allergen in No. 1 vial is 1/100 dilutions of No. 3 vials used for maintenance treatment and the concentration of allergen in No. 2 vial is 1/10 dilutions of No. 3 vials. The injection of above allergen solution for allergen-immunotherapy was subcutaneously administered with sequentially increased volumes of 0.1 ml, 0.2 ml, 0.4 ml, and 0.8 ml of No. 1 vial and then 0.1 ml, 0.2 ml, 0.4 ml, and 0.8 ml of No. 2 vial and then 0.1 ml, 0.2 ml, 0.4 ml, and 0.8 ml of No.3 vial at a weekly interval for 12 weeks and then 0.8 ml of No. 3 vial at a monthly interval.

### Example of Formulation 2-2: Histamine-Immunoglobulin Complex

An injection formulation for histamine-mmunoglobulin complex (HISTOBULIN™, Green cross PBM, Korea) contained 12 mg of human immunoglobulin and 0.15 μg of histamine dichloride as described in the information of the product provided by the manufacturer. The above formulation is provided as in two vials comprising one vial containing the above active ingredients in a lyophilized form and another vial containing 2 ml of distilled water for injection and the manufacturer recommends to mix the contents of two vials using an injection syringe immediately before the each injections and dissolve the active ingredients well and inject 2 ml of this mixture subcutaneously. When the contents of IgG, IgM, IgA and albumin concentration in the above histamine-immunoglobulin complex injection solution were determined by nephelometry analyzer (COBAS INTEGRA, Roche Diagnostics GmbH, Germany), the 11.0 mg of IgG and 0.24 mg of IgA were contained in the above formulation, but IgM or albumin was not detected in the above formulation because their concentrations were below the lowest detection limits of the above analyzer.(IgM <0.037 mg/ml, albumin <0.09 mg/ml).

### Example 1

Clinical trial examples showing that the house dust mite-allergic patients having both respiratory allergic diseases and atopic dermatitis simultaneously and received a standard allergen-immunotherapy but did not show improvement of clinical symptoms of atopic dermatitis and then these patients were markedly improved after change to a combination treatment of allergen-immunotherapy and histamine-immunoglobulin complex.

### Clinical Trial Example 1

A 24-year old female patient visited the outpatient clinic for symptoms including severe itching, dryness of skin, scaling of skin, eczema involving skin of the whole body including face and skin behind the ear, rhinorrhea, sneezing, and nasal obstruction lasted for the past 8 years. The patient complained that her usual daily life had markedly been interfered by the above clinical symptoms. Although the patient continuously received various medical treatments at many local clinics, she did not experience any clear improvements in her clinical symptoms. The patient showed a strong positive reaction to two kinds of house dust mites (D. pteronyssinus and D. farinae) with the mean wheal diameters over 6 mm on the allergy skin prick test and the concentration of serum specific IgE antibodies to house dust mite (D. pterontyssinus) was elevated to 66.7 kU/L and serum total IgE level also was elevated to above the 1000 IU/ml. Accordingly, she was clinically diagnosed as atopic dermatitis and allergic rhinitis with house dust mite allergy. And then the above patient received oral antihistamine, nasal spray of corticosteroid and also received house dust mite allergen-immunotherapy using the above example of formulation 2-1 (Novo-Helisen Depot™) as described. Even 9 months after she received maintenance treatment of subcutaneously injecting 0.8 ml of maintenance concentration of house dust mite extracts at a monthly interval, any significant improvements of clinical symptoms of atopic dermatitis and allergic rhinitis could not be observed. Then she was subcutaneously injected a direct mixture of lyophilized powder containing histamine-immunoglobulin complex described in the above example of formulation 2-2 (HISTOBULIN™, Green cross PBM, Korea; a vial containing lyophilized powder comprising 12 mg of human immunoglobulin and 0.15 μg of histamine dichloride) with injection solution of the above house dust mite allergen-immunotherapy at a monthly interval. Her clinical symptoms severity of atopic dermatitis and allergic rhinitis began to markedly improve at 3 months after the above combination treatment. At the 9 months after the above combination treatment these clinical severity of atopic dermatitis and allergic rhinitis were significantly improved more than 50% compared to the baseline clinical severity before the start of the above combination treatment of house dust mite allergen-immunotherapy and histamine-immunoglobulin complex judged by both the patient's own subjective assessment and the physician's objective assessment based on the physical examination of patient. The extent of dermatitis and severity of dermatitis and degree of skin scaling were improved more than 50% compared to the baseline status before the above combination treatment and her symptoms of allergic rhinitis also improved and she did not experienced rhinorrhea and sneezing even though she did not take oral antihistamine or nasal corticosteroid spray anymore.

### Clinical Trial Example 2

A 30-year old female patient visited the outpatient clinic for coughing aggravated at night, wheezing, difficult breathing, rhinorrhea, sneezing, itching of eyeball, itching of whole body, and dryness of skin involving whole body. Although the patient continuously received various medical treatments for the past 5 years at many local clinics, she did not experience any clear improvements in her clinical symptoms. The patient showed strong positive reactions to two kinds of house dust mites (D. pteronyssinus and D. farinae) with the mean wheal diameters over 6 mm on the allergy skin prick test and the concentrations of serum specific IgE antibodies to two kinds of house dust mites (D. pteronyssinus and D. farinae) were elevated above the 3.5 kU/L and showed positive result on the methacholine bronchial challenge test (20% decrease in forced expiratory volume in one second following the inhalation of less than 8 mg methacholine/ml). Accordingly, she was clinically diagnosed as allergic asthma, allergic rhinitis, allergic conjunctivitis, and a topic dermatitis with house dust mite allergy. And then the above patient received oral antihistamine, oral leukotriene-antagonist, daily inhalation of corticosteroid and long acting beta-2 agonist (flixotide+salmeterol) in a combined formulation. Her clinical symptoms of allergic asthma and rhinitis were partially improved but still experienced intermittent coughing and sputum after 6 months of the above pharmacological treatment. After explaining about the allergen-immunotherapy to the patient, she received house dust mite allergen-immunotherapy using the above example of formulation 2-1 (Novo-Helisen Depot™) as described. When she was examined 24 months after she received maintenance treatment subcutaneously injecting 0.8 ml of maintenance concentration of house dust mite extracts monthly, her clinical symptoms of respiratory allergic diseases including coughing, difficult breathing, rhinorrhea, and sneezing were significantly improved but clinical symptoms associated with atopic dermatitis including itching of whole body, scaling of skin, dryness of skin involving whole body, and reddish itching eruptions on face were not improved compared those before the start of house dust mite allergen-immunotherapy. Then she was subcutaneously injected a direct mixture of lyophilized powder containing histamine-immunoglobulin complex described in the above example of formulation 2-2 (HISTOBULIN™, Green cross PBM, Korea) with injection solution of the above house dust mite allergen-immunotherapy monthly. Her clinical symptom of atopic dermatitis including skin itching, dryness of skin, and scaling of skin and symptoms related to allergic rhinitis began to markedly improve after 3 months of starting the above combination treatment. At the 6 months after the above combination treatment, the clinical severity of atopic dermatitis were significantly improved more than 50% compared to the baseline before the start of the above combination treatment of house dust mite allergen-immunotherapy and histamine-immunoglobulin complex judged by both the patient's own subjective assessment and the physician's objective assessment based on the physical examination of the patient. At the 12 months after the above combination treatment the clinical severity of atopic dermatitis were significantly improved more than 80% compared to the baseline clinical severity before the start of the above combination treatment judged by both the patient's own subjective assessment and the physician's objective assessment based on the physical examination of the patient.

### Clinical Trial Example 3

A 26-year old female patient visited the outpatient clinic for rhinorrhea, sneezing, itching of whole body, scaling of skin, and dryness of skin involving whole body lasted for the past 10 years. The patient showed strong positive reactions to two kinds of house dust mites (D. pteronyssinus and D. farinae) with the mean wheal diameters over 6 nm on the allergy skin prick test and the concentrations of serum specific IgE antibodies to two kinds of house dust mites (D. pteronyssinus and D. farinae) were elevated above the 3.5 kU/L. Accordingly, she was clinically diagnosed as atopic, dermatitis and allergic rhinitis with house dust mite allergy. And then the above patient received oral antihistamine, topical corticosteroid treatment, and also received house dust mite allergen-immunotherapy using the above example of formulation 2-1 (Novo-Helisen Depot™) as described. When the patient was examined at 36 months after she received maintenance allergen-immunotherapy treatment of subcutaneously injecting 0.8 ml of maintenance concentration of house dust mite extracts monthly, her clinical symptoms of allergic rhinitis including rhinorrhea and sneezing were significantly improved more than 70% compared to those before the start of allergen-inmunotherapy but clinical symptoms of atopic dermatitis including itching of whole body, scaling of skin, dryness of skin, and eczema of scalp were not significantly improved compared those symptoms before the start of allergen-immunotherapy. Then she was subcutaneously injected a direct mixture of lyophilized powder containing histamine-immunoglobulin complex described in the above example of formulation 2-2 (HISTOBULIN™, Green cross PBM, Korea) with injection solution of the above house dust mite allergen-immunotherapy monthly. Her clinical symptom of atopic dermatitis including skin itching, dryness of skin, and scaling of skin and symptoms related to allergic rhinitis began to markedly improve after 2 months of starting the above combination treatment. At the 12 months after the above combination treatment the clinical severity of atopic dermatitis were significantly improved more than 50% compared to the baseline before the start of the above combination treatment of house dust mite allergen-immunotherapy and histamine-immunoglobulin complex judged by both the patient's own subjective assessment and the physician's objective assessment based on the physical examination of the patient.

The above 3 clinical trial examples confirm that the present invention can provide a new improved pharmacological composition and an effective treatment method using the composition that can induce a improvements of atopic dermatitis that is not improved by traditional standard allergen-immunotherapy in patients with allergic diseases and house dust mite allergy.

### [Example 2

The therapeutic efficacy of combination treatment with allergen-immunotherapy and histamine-immunoglobulin complex in a patient having atopic dermatitis, allergic rhinitis, and allergic asthma simultaneously.

### Clinical Trial Example

A 24-year old female patient visited the outpatient clinic for itching of whole body, dryness of skin, scaling of skin, and eczema involving whole body lasted for 10 years and recently aggravated 4 months ago. She could not sleep properly due to the above symptoms. On the medical history examination, she was experiencing symptoms of allergic rhinitis including rhinorrhea and sneezing and also experiencing symptoms of bronchial asthma including intermittent coughing, difficult breathing, and wheezing. The patient showed strong positive reactions to two kinds of house dust mites (D. pteronyssinus and D. farinae) with the mean wheal diameters over 5 mm on the allergy skin prick test and the concentrations of serum specific IgE antibodies to two kinds of house dust mites (D. pteronyssinus and D. farinae) were 5.07 kU/L (D. pteronyssinus—specific IgE) and 6.87 kU/L (D. farinae—specific IgE) respectively and the serum total IgE concentration was 58 IU/ml. Accordingly, she was clinically diagnosed as atopic dermatitis, allergic rhinitis, and allergic asthma with house dust mite allergy. And then the above patient started to receive oral antihistamine and low dose oral corticosteroid treatment, and also received house dust mite allergen-immunotherapy using the above example of formulation 2-1 (Novo-Helisen Depot™) as described and she also received histamine-immunoglobulin complex described in the above example of formulation 2-2 (HISTOBULIN™, Green cross PBM, Korea) directly mixed with the above allergen-immunotherapy solution at every injections of allergen-immunotherapy. Her clinical symptom of atopic dermatitis, allergic rhinitis, and allergic asthma began to markedly improve after 1 month of starting the above combination treatment. At the 3 months after the above combination treatment, the clinical severity of atopic dermatitis, allergic rhinitis, and allergic asthma were significantly improved more than 70% ccmpared to the baseline before the start of the above combination treatment of house dust mite allergen-immunotherapy and histamine-innnunoglobulin complex judged by both the patient's own subjective assessment and the physician's objective assessment based on the physical examination of the patient. Especially, her clinical features of atopic dermatitis including the involved area of atopic dermatitis and severity of eczema were significantly improved more than 70% compared to the baseline before the start of the above combination treatment. She did not experienced any, difficulties in the daily living with oral antihistamine treatment alone and did not need to receive oral corticosteroid anymore and her all kinds of clinical symptoms of allergic diseases were significantly improved. These clinical efficacies were lasted even at 6 months and 12 months after the start of the above combination treatment.

### Example 3

Clinical trial examples comparing the clinical efficacy and side effect of 3 different treatment methods including allergen-immunotherapy, histamine-immunoglobulin complex, and a combination treatment of allergen-immunotherapy and, histamine-immunoglobulin complex in house dust mite-sensitized patients with refractory atopic dermatitis

Patients with atopic dermatitis, who fulfill all of the below criteria were included.

The patients showed the typical clinical features of atopic dermatitis including chronic persisting or frequently recurring itching of skin, dryness of skin, scaling of skin, and typically distributing eczermatous skin lesions and met the diagnostic criteria for the atopic dermatitis suggested by Hanifin and Rajka (Hanifin J M, Rajka G. Acta Derm Venereol (Stockh) 1980;92 (Suppl.):44-47). They also showed positive reactions (mean wheal diameter over 3 mm) to two kinds of house dust mites (D. pteronyssinus and D. farinae) on allergy skin prick test or positive results on tests for serum specific IgE antibodies to two kinds of house dust mites (D. pteronyssinus and D. farinae) as judged by the criteria made by manufacturer of specific IgE tests. Among the above patients meeting the inclusion criteria, patients with refractory atopic dermatitis who were not improved sufficiently to be satisfied by himself or herself even after receiving standard treatments including topical corticosteroids, topical moisturizers, and oral antihistamines were included in the 3 treatment methods described in the below.

Group 1 (standard allergen-immunotherapy group): The house dust mite allergen-immunotherapy of the above example of formulation 2-1 (Novo-Helisen Depot™) was administered as recommended by manufacturer.

Group 2 (histamine-immunoglobulin complex group): The histamine-immunoglobulin complex of the above example of formulation 2-2 (HISTOBULIN™, Green cross PBM, Korea) was administered with a slightly modifications of the intervals for administration recommended by manufacturer.

The above formulation was administered subcutaneously every one week for 8 weeks and then two times at 2 weeks interval from 8-12 weeks and monthly interval after 12 weeks.

Group 3 (a combination treatment of allergen-immunotherapy and histamine-immunoglobulin complex): The treatment methods of group 1 and group 2 were simultaneously administered. To minimize the inconvenience of patients for injecting the two kinds of formulation separately in both arms, the injection solution for histamine-innnunoglobulin complex (2 ml) was directly mixed with injection solution for house dust mite allergen-immunotherapy (minimal 0.1 ml to maximal 0.8 ml) using injection syringe and subcutaneously injected simultaneously to one arm with maximal 2.8 ml volume of injection. The above injection method was well tolerated by the all patients and patients complained least of the pain due to injection compared to the injecting two kinds of formulation separately in both arms. And inventors found that methods of mixing the above two kinds of injection solutions and administrating simultaneously were very safe and could relieve the patients from the pains due to multiple injections. When the house dust mite allergen-immunotherapy reached the maintenance schedule at 12 weeks after the start of allergen-immunotherapy, the allergen solution (0.8 ml of No. 3 vial) was directly mixed to the lyophilized powder containing histamine-immunoglobulin complex and the mixture was rapidly dissolved and the 0.8 ml of injection solution containing both house dust mite allergen and histamine-immunoglobulin complex was injected at a monthly interval.

Oral antihistamines and topical corticosteroids were maintained as before the start of above treatments in all three treatment groups.

The clinical improvement of atopic dermatitis was evaluated by combining the two criteria as described below.

(1) Subjective assessment of clinical improvement obtained by medical history of patients: The patients were asked to tell the subjective improvement of clinical symptoms related to atopic dermatitis including itching, dryness, scaling, and eczema at monthly interval from the beginning of treatment. Patients were educated to express the improvement as 100% if their symptcms were completely disappeared and 0% if their symptoms were not least improved or even aggravated and 50% if their symptoms were decreased to half of baseline. The subjective improvements assessed by patients were recorded monthly and analyzed.

(2) Global assessment of general severity of atopic dermatitis on the basis of physical examination by physician: The physician involved in the patient care measured the clinical severity of atopic dermatitis (comprehensively assessing degrees of scaling, eczema, thickening of skin, and area of involvement) by physical examination of patients at a monthly interval using the investigator's global assessment (IGA) criteria as previously described (Gelmetti C, et al. Allergy 2004;59(Suppl. 78):61-65). The IGA scoring was made very severe (5), severe (4), moderate (3), mild (2), almost clear (1), and clear (0). The scoring was made monthly and recorded.

The above patients were continued the treatments for more than 1 year. The clinical improvement was analyzed using the mean improvement values calculated from the summation of improvements assessed by the above two methods. Assessment of clinical improvement was analyzed at 6 months, 12 months after the start of the above treatments compared to the clinical severity before the start of the above treatments (Table 2). To evaluate the long-term clinical efficacy of the above treatments, the results from the patients who received the above treatments continuously for more than 6 months were analyzed.

TABLE 1
Characteristics of treatment groups (S.D. means standard deviation)

| | Patients | Sex | Age |
|---|---|---|---|
| Treatment groups | No. | (F/M) | (Mean ± S.D.) |
| Group 1 | 15 | 8/7 | 22.5 ± 9.1 |
| (standard allergen-immunotherapy) | | | |
| Group 2 | 10 | 3/7 | 20.9 ± 4.6 |
| (histamine-immunoglobulin complex) | | | |
| Group 3 | 21 | 9/12 | 23.7 ± 1.1 |
| (combination treatment of | | | |
| allergen-immunotherapy | | | |
| and histamine-immunoglobulin | | | |
| complex) | | | |

TABLE 2
Comparing the clinical improvements between the treatment groups

| | Clinical improvement | | Clinical improvement | |
|---|---|---|---|---|
| | after 6 months | | after 12 months | |
| | Improvement | Improvement | Improvement | Improvement |
| | more than | more than | more than | more than |
| Treatment groups | 20% | 50% | 20% | 50% |
| Group 1 | 7/15 (47%) | 0/15 (0%) | 8/12 (67%) | 1/12 (8.3%) |
| (standard | | | | |
| allergen-immunotherapy) | | | | |
| Group 2 | 7/10 (70%) | 1/10 (10%) | 6/9 (67%) | 1/9 (11%) |
| (histamine-immunoglobulin | | | | |
| complex) | | | | |
| Group 3 | 19/21 (91%)* | 11/21 (52%)† | 19/20 (95%) | 18/20 (90%)‡ |
| (combination treatment of | | | | |
| allergen-immunotherapy and | | | | |
| histamine-immunoglobulin | | | | |
| complex) | | | | |

*Fisher's exact test P < 0.01, when comparing the frequency of improvement more than 20% with group 1.
†Fisher's exact test P < 0.05, when comparing the frequency of improvement more than 50% with group 1 or group 2.
‡Fisher's exact test P < 0.001, when comparing the frequency of improvement more than 50% with group 1 or group 2.
When the patients were followed up for 12 months, 3 patients of group 1 and 1 patient of group 2 and 1 patient of group 3 were lost from the follow-up.

From the analyzed results of the above (Table 2), it could be confirmed that marked clinical improvements more than 50% compared to clinical severity before the start of the above treatments were more frequently observed in the patient group treated by the above combination treatment method of the presents invention (group 3 in Table 2) compared to the patient groups treated by allergen-immunotherapy alone or histamine-immunoglobulin complex alone at 6 months or 12 months after start of the above treatments (p<0.05, Fisher's exact test). In group 1 patients treated by standard allergen-immunotherapy, one patient experienced generalized urticaria and two patients experienced severe edema of injection site. No case of local or generalized side effect was observed in the group 2 patients or group 3 patients. The majorities of patients with atopic dermatitis (more than 90% of patients) in the above three treatment groups also experienced the clinical symptoms of allergic rhinitis. The clinical symptoms of allergic rhinitis were improved in the more than 50% compared to those symptoms before the start of the above treatment in more than 80% of patients who received three kinds of treatment when determined by medical history at 6 months and 12 months after the start of the above treatment. And these results indicate that a combination of allergen-immunotherapy and non-specific immunotherapy using histamine-immunoglobulin complex of the present invention is a safe treatment without side effects. Because the immunoglobulin contained in the present invention is not selectively including the allergen-specific antibodies, the present invention provides a pharmaceutical composition that can be more easily produced, needs lower costs for production, and provides higher safety of the product compared to the pharmaceutical composition comprising immune complex of allergen and allergen-specific antibodies as designed by Saint-Remy (Saint-Remy J M, et al. Clin Exp Allergy 1994;24:1091-3). The present invention also provides an advanced pharmaceutical formulation and an advanced treatment method applicable for the allergen-immunotherapy of allergic diseases that can be easily used by physicians due to fewer treatment-related side effects. Because the present invention is using the same techniques that is used for the production of pharmaceutical formulation for allergen-immunotherapy and histamine-immunoglobulin complex and manufacturing techniques for those are highly developed already, the present invention provides an pharmacological composition that has higher effectiveness, fewer side effects, higher safety, and can be easily manufactured compared to current standard therapeutic drugs. Therefore, the pharmaceutical composition of the present invention can provide an opportunity for clinical improvements to the large numbers of patients with allergic diseases.

### Example 4

Clinical trial examples of the patients having respiratory allergic diseases and showing allergic reaction to pollens and house dust mites simultaneously and experienced systemic side effects during the allergen-immunotherapy, but subsequently improved by a treatment with a pharmaceutical composition of the present invention with minimizing the side effects.

### Clinical Trial Example 1

A 28-year old female patient visited the outpatient clinic for coughing aggravated at night, wheezing, difficult breathing, rhinorrhea, sneezing, and chronic intermittent urticaria lasted for past 2 years. The patient showed strong positive reactions to two kinds of house dust mite (D. pteronyssinus and D. farinae) and mugwort pollen with the mean wheal diameters over 6 inn on the allergy skin prick test and the tests of serum specific IgE antibodies to the above three allergens were positive and showed positive result on the methacholine bronchial challenge test (20% decrease in forced expiratory volume in one second following the inhalation of less than 8 mg methacholine/ml). Accordingly, she was clinically diagnosed as allergic asthma, allergic rhinitis, and chronic urticaria with allergy to house dust mites and mugwort pollen. And then the above patient received oral antihistamine, oral leukotriene-antagonist, daily inhalation of corticosteroid and long acting beta-2 agonist (budesonide+formoterol) in a combined formulation and her clinical symptoms of allergic asthma, allergic rhinitis, and chronic urticaria were partially improved, but she still experienced intermittent coughing, sputum and urticaria after 6 months of the above pharmacological treatment. After explaining about the allergen-immunotherapy to the patient, she received allergen-immunotherapy using the allergen extract formulation (Novo-Helisen Depot™; Allergopharma, Germany; injection solution for maintenance therapy schedules contained 60-80 g/ml of protein quantified by Bradford's method and also contained aluminum hydroxide, 0.4% phenol, and saline solution) containing 25% of D. pteronyssinus and 25% of D. farillae and 50% of mugwort pollen. The injection of above allergen solution for allergen-irnnmnotherapy was subcutaneously administered as reconmmended by the manufacturer to reach the final maintenance Use at 12 weeks by sequentially increasing the allergen concentration in injection. When she was subcutaneously injected with the 0.4 ml of the final allergen concentration (No. 3 vial), she experienced severe generalized urticaria at the same day of allergen injection. So, the allergen dose for injection was reduced to 0.2 ml of the final allergen concentration and administered at monthly interval, but her clinical symptoms of allergic asthma, allergic rhinitis, and intermittent urticaria were not improved and persisted. Then she was subcutaneously injected a mixture of 0.4 ml of the above allergen solution of final maintenance concentration and 2 ml of solution containing histamine-immunoglobulin complex described in the above example of formulation 2-2 (HISTOBULIN™, Green cross PBM, Korea) but she did not experienced generalized urticaria. Then she was administered 0.5 ml of the above allergen solution with 2 ml of solution containing histamine-immunoglobulin complex at 1 month later and the amounts of allergen solution was further increased to 0.6 ml after 2 months later and she did not experienced any local or systemic side effects due to injection of the above mixtures. At the 3 months after the above combination treatment, the clinical symptoms of allergic asthma, allergic rhinitis, and chronic urticaria were significantly improved and maintained the improvement state without treatments with oral administration of drugs.

### Clinical Trial Example 2

A 12-year old male patient visited the outpatient clinic for chronic recurrent rhinorrhea, sneezing, nasal obstruction, itching of eyeball, and congestion of conjunctiva with clinical symptoms compatible with allergic rhinitis and allergic conjunctivitis. The patient showed strong positive reactions to two kinds of house dust mites (D. pteronyssinus and D. farinae), mugwort pollen, and dandelion pollen with the mean wheal diameters over 6 nm on the allergy skin prick test and the tests of serum specific IgE antibodies to the above four allergens were positive (elevated above the 3.5 kU/L). Accordingly, he was clinically diagnosed as allergic rhinitis and allergic conjunctivitis with allergy to house dust mite, mugwort pollen, and dandelion pollen. And then the above patient received oral antihistamine, oral leukotriene-antagonist, and nasal spray of corticosteroid daily. His clinical symptoms of allergic rhinitis and allergic conjunctivitis were partially improved. The parents of the patient wanted more fundamental treatment. So, he received allergen-immunotherapy using the allergen extract formulation (Novo-Helisen Depot™; Allergopharma, Germany) containing 30% of D. pteronyssinus, 20% of D. farinae, 30% of mugwort pollen, and 20% of dandelion pollen after explaining about the effect and side effects of allergen-imnunotherapy to parents of the patient. The injection of above allergen solution for allergen-inmunotherapy was subcutaneously administered as reca ended by the manufacturer to reach the final maintenance dose at 12 weeks by sequentially increasing the allergen concentration of injection. When he was subcutaneously injected with the 0.8 ml of the final allergen concentration (No. 3 vial), he experienced severe generalized urticaria and breathing difficulty at the same day of allergen injection and he visited emergency room for the treatment of the above symptoms. So, the allergen dose for injection was reduced to 0.5 ml of the final allergen concentration and administered at monthly intervals, but his clinical symptoms of allergic rhinitis and allergic conjunctivitis were not improved and persisted. Then he was subcutaneo usly injected a direct mixture of 0.8 ml of the above allergen solution of final maintenance concentration and dry powder containing histamine-immunoglobulin complex described in the above example of formulation 2-2 (HISTOBULIN™, Green cross PBM, Korea) but he did not experienced any side effects including generalized urticaria. Then he was administered 0.8 ml of the above allergen solution mixed with histamine-immunoglobulin complex at monthly interval. At the 3 months after the above combination treatment, the clinical symptoms of allergic rhinitis and allergic conjunctivitis including rhinorrhea, sneezing, itching of eyeball, and congestion of conjunctiva were significantly improved more than 50% compared to the baseline before the start of the allergen-immunotherapy.

The above clinical trial examples confirm that a pharmacological composition of the present invention and a treatment method using the above composition combining allergen-immunotherapy and nonspecific immunotherapy using histamine-immunoglobulin complex can provide a clinically more safe treatment method that can inhibit the developments of systemic side effects induced by the traditional standard allergen-immunotherapy. The above clinical trial examples also confirm that a treatment method of the present invention is useful for not only patients with allergic diseases having house dust mite allergy but also patients with allergic disease having allergy to pollens. The above clinical trial examples also confirm that a pharmacological composition of the present invention comprising allergen, histamine, and immunoglobulin and a treatment method using the above composition is effective for not only atopic dermatitis but also allergic conjunctivitis, allergic asthma, allergic rhinitis, and urticaria.

### Example 5

Analysis of the clinical efficacy of a pharmacological composition of the present invention and a treatment method of the present invention by measuring the disease severity of atopic dermatitis using a standardized severity scoring index (SCORAD index) in patients, with refractory atopic dermatitis

Patients with refractory atopic dermatitis who were not sufficiently improved by standard pharmacological treatments and topical moisturizing therapy and who showed positive reactions to house dust mites on allergy skin prick test and positive results on tests for serum specific IgE antibodies to house dust mites were included in this trial. The above patients with refractory atopic dermatitis received a combination treatment of the present invention as described in the group 3 of the above Example 3. The clinical efficacy of the above combination treatment of the present invention was analyzed by evaluating the clinical severity of atopic dermatitis before the start of the above combination treatment, and at 6 months and 12 months after the starting the above treatment using a standardized clinical severity scoring index for atopic dermatitis (SCORAD index; European task force on atopic dermatitis. Dermatology 1993;186:23-31) that is most widely used in the recent international studies on the atopic dermatitis (Table 3, Table 4).

TABLE 3
Characteristics of patients in treatment group (number of patients = 14)

| | | Difference of |
|---|---|---|
| | | SCORAD index |
| | SCORAD index | before and after treatment |
| | Mean ± S.D. | Mean ± S.D. |
| Before treatment | 45.3 ± 17.2 | |
| 6 months after treatment | 30.8 ± 19.0* | −14.4 ± 1.8 |
| 12 months after treatment | 19.0 ± 15.5* | −26.3 ± 1.9 |

*p < 0.005 compared to the SCORAD index of baseline (before treatment) by Wilcoxon Signed Rank test.
S.D. = standard deviation.

TABLE 4
Analysis of clinical efficacy of a combination treatment of the present
invention in patients with severe atopic dermatitis (baseline SCORAD
index being 40 or more) (number of patients = 8)

| | | Difference of |
|---|---|---|
| | | SCORAD index |
| | SCORAD index | before and after treatment |
| | Mean ± S.D. | Mean ± S.D. |
| Before treatment | 56.5 ± 11.6 | |
| 6 months after treatment | 39.9 ± 20.0* | −16.7 ± 4.5 |
| 12 months after treatment | 27.3 ± 14.8* | −29.2 ± 3.6 |

*p < 0.05 compared to the SCORAD index of baseline (before treatment) by Wilcoxon Signed Rank test.
S.D. = standard deviation.

The above table 3 and table 4 show that a combination treatment of the present invention significantly improved the clinical severity of atopic dermatitis compared to the baseline clinical severity of atopic dermatitis before the start of the above treatment when analyzed using the SCORAD index. Especially, when the patients with severe atopic dermatitis showing SCORAD index being the same or greater than 40 were analyzed, the combination treatment of the present invention induced greater clinical improvement at the 12 months after the start of the treatment (mean decrease of SCORAD index was 29; table 4) compared to the recent report on the clinical efficacy of house dust mite allergen-immunotherapy in patients with severe atopic dermatitis having house dust mite allergy and SCORAD index being the same or greater than 40 (mean decrease of SCORAD index was 19) (Werfel T, et al. Allergy 2006;61:202-205). And as shown in table 2, a combination treatment of the present invention showed that 20 patients among the 21 patients with refractory atopic dermatitis treated with the combination treatment of the present invention continued the treatment for 1 year (compliance rate 95%) and this result was significantly better than the results of the recent report showed that the house dust mite allergen-immunotherapy was effective for the treatment of severe atopic dermatitis and only 18 patients among 33 patients with atopic dermatitis initially included finished the allergen-immunotherapy for 12 months (compliance rate 55%) in that report (Werfel T, et al. Allergy 2006;61:202-205).

### Example 6

Examples that confirmed whether the therapeutic effect of the combination of allergen-immunotherapy and histamine-immunoglobulin complex is simply an additive effect of two therapeutic compositions or a synergistic effect produced by the combination of two therapeutic compositions.

### Clinical Trial Example 1

A 27-year old female patient visited the outpatient clinic for symptoms including rhinorrhea, sneezing, severe itching of skin involving the whole body, scaling of skin, dryness of skin, eczematous eruptions involving skin of the face lasted for the past 20 years. The patient showed a strong positive reaction to two kinds of house dust mites (D. pteronyssinus and D. farinae) with the mean wheal diameters over 6 mm on the allergy skin prick test and the concentrations of serum specific IgE antibodies to two kinds of house dust mites (D. pteronyssinus and D. farinae) were elevated above the 3.5 kU/L and showed positive results. Accordingly, she was clinically diagnosed as allergic rhinitis and atopic dermatitis with house dust mite allergy. Although the patient continuously received various medical treatments for the past 10 years at many clinics, she did not experience any clear improvements in her clinical symptoms. The patient refused to take any oral medication. So, she was only received topical corticosteroids. And then the patient received house dust mite allergen-immunotherapy using the above example of formulation 2-1 (Novo-Helisen Depot™) as described in the group 1 of the above Example 3 and she also received histamine-immunoglobulin complex described in the above example of formulation 2-2 (HISTOBULIN™, Green cross PBM, Korea) as described in the group 2 of the above Example 3. The two treatments were separately administered to the 2 different arms at the same intervals described in the group 1 and group 2 treatment of the above Example 3. At the 6 months after the above treatments, the clinical symptoms including rhinorrhea, sneezing, itching of skin involving the whole body, scaling of skin, dryness of skin, eczematous eruptions involving skin of the face were not significantly improved compared to the clinical symptoms before the start of the above treatment judged by the physician's objective assessment based on the physical examination of patient and the patient also complained about lack of any clinical improvement by the above treatments. Then she was subcutaneously injected a direct mixture of lyophilized powder containing histamine-immunoglobulin complex described in the above example of formulation 2-2 (HISTOBULIN™, Green cross PBM, Korea) with 0.8 ml of injection solution for house dust mite allergen-immunotherapy of the above formulation at once. One week after the start of the above combination treatment, her clinical symptoms including skin itching, dryness of skin, and scaling of skin, eczematous eruptions involving skin of the face, rhinorrhea, and sneezing suddenly began to improve. At one month after the start the above combination treatment of histamine-immunoglobulin complex and allergen immunotherapy, her clinical symptoms related to atopic dermatitis and allergic rhinitis were significantly improved more than 50% compared to before the start of the above combination treatment judged by both the patient's own subjective assessment and the physician's objective assessment based on the physical examination of the patient. The above result indicates that the treatment of the present invention combining allergen-immunotherapy and histamine-immunoglobulin complex is not simply an additive effect of the two therapeutic compositions but a synergistic effect produced by the combination of two therapeutic compositions.

### Clinical Trial Example 2

A patient with atopic dermatitis was treated by the combination of house dust mite allergen-immunotherapy and histamine-immunoglobulin complex as described in the group 3 of the above Example 3 of the present invention and the patient's clinical symptoms related to atopic dermatitis was improved more than 50% compared to the baseline clinical severity before the start of the above combination treatment as judged by both the patient's own subjective assessment and the physician's objective assessment based on the physical examination of patient after receiving monthly maintenance treatments of the above combination treatment for more than 6 months. The patient wanted to receive house dust mite allergen-immunotherapy only without histamine-immunoglobulin complex and he was administered the same dose of house dust mite allergen-immunotherapy at the same interval. At 2 months after changing to house dust mite allergen-immunotherapy alone, the patient's clinical severity of atopic dermatitis was evidently aggravated to the level before the start of the above combination treatment. Then the patient received the combination of house dust mite allergen-immunotherapy and histamine-immunoglobulin complex as described in the group 3 of the above Example 3 of the present invention again. At one month after returning to the above combination treatment, the atopic dermatitis began to improve. At the 3 months after the returning to the above combination treatment, the clinical features related to atopic dermatitis were significantly improved more than 50% compared to the clinical severity before the starting of the above combination treatment and the improvement was maintained.

### Clinical Trial Example 3

Two patients with atopic dermatitis were treated by the combination of house dust mite allergen-immunotherapy and histamine-immunoglobulin complex as described in the group 3 of the above Example 3 of the present invention and clinical symptoms related to atopic dermatitis of the two patients were improved more than 50% compared to the baseline clinical severity before the start of the above combination treatment as judged by both the patient's own subjective assessment and the physician's objective assessment based on the physical examination of patient after receiving monthly maintenance treatments of the above combination treatment for more than 6 months. After getting the permission of two patients, the commercially available human gammaglobulin for injection (Green cross PBM, Korea; contains 165 mg/ml of human gammaglobulin according to the product information provided by manufacturer; nephelometric measurement of this product showed that the product contained IgG 150 mg/ml, IgA 0.14 mg/ml, IgM<0.04 mg/ml, and albumin 1.58 mg/ml) or human IgG for intravenous administration (Green cross PBM, Korea; contains 50 mg/ml of human IgG according to the product information provided by manufacturer; nephelometric measurement of this product showed that the product contained IgG 50.92 mg/ml, IgA<0.013 mg/ml, IgM<0.04 mg/ml, and albumin<0.09 mg/ml) was diluted by saline and a portion of human gammaglobulin or human IgG was sampled to contain 12 mg of human IgG and mixed with 0.8 ml of the above injection solution for house dust mite allergen-immunotherapy and administered to the above two patients (one patient received human gammaglobulin and the other patient received human IgG) in a monthly interval. At one month after changing to the above treatment, clinical symptoms and objective findings of atopic dermatitis were evidently aggravated more than 30% compared to before the start of the above combination treatment in the two patients. At two months after changing to the above treatment, the clinical symptoms and objective findings of atopic dermatitis were even more aggravated. Then the two patients received the combination of house dust mite allergen-immunotherapy and histamine-immunoglobulin complex as described in the group 3 of the above Example 3 of the present invention again. At two months after returning to the above combination treatment, the atopic dermatitis began to significantly improve. At the 3 months after the returning to the above combination treatment, the clinical features related to atopic dermatitis were significantly improved more than 50% compared to the baseline clinical severity before the start of the initial treatment of the above combination treatment and the improvement was maintained in the above two patients.

### Clinical Trial Example 4

A patient with atopic dermatitis was treated by the combination of house dust mite allergen-immunotherapy and histamine-immunoglobulin complex as described in the group 3 of the above Example 3 of the present invention and the patient's clinical symptoms related to atopic dermatitis was improved more than 50% compared to the baseline clinical severity before the start of the above combination treatment as judged by both the patient's own subjective assessment and the physician's objective assessment based on the physical examination of patient after receiving monthly maintenance treatments of the above combination treatment for more than 6 months. After getting the permission of the patient, house dust mite allergen-immunotherapy and histamine-innnunoglobulin complex in maintenance doses were separately administered to the 2 different arms at monthly interval. At two months after changing to the above treatment, clinical symptoms and objective findings of atopic dermatitis were evidently aggravated more than 30% compared to before the changing to the above treatment. Then the patient received the combination of house dust mite allergen-immunotherapy and histamine-immunoglobulin complex as described in the group 3 of the above Example 3 of the present invention again. At one month after returning to the above combination treatment, the atopic dermatitis began to significantly improve. At the 3 months after the returning to the above combination treatment, the clinical features related to atopic dermatitis were significantly improved more than 50% compared to the baseline clinical severity before the start of the initial treatment of the above combination treatment and the improvement was maintained.

The above clinical trial examples confirm that the therapeutic efficacy of a pharmaceutical composition and a treatment method of the present invention combining allergen-immunotherapy and histamine-immunoglobulin complex is not simply an additive effect of the two therapeutic compositions but a synergistic effect produced by combination of two therapeutic compositions.

The presence of house dust mite-specific IgG and IgA antibodies in the blood of normal subjects has been reported (Hong C S, et al. Yonsei Med J 1994;35:453-63; Stewart G A, et al. Clin Allergy 1988;18:235-43; Saint-Remy J M, et al. Allergy 1988;43:338-47). On the basis of the above reports, the therapeutic efficacy of the present invention combining allergen-immunotherapy and histamine-immunoglobulin complex might be attributed to formation of immune complex made of house dust mite allergen and house dust mite allergen-specific IgG antibodies contained in the histamine-immunoglobulin complex. However, as shown in the above clinical trial example 3, the therapeutic efficacy of the above combination of the present invention was disappeared when the histamine-immunoglobulin complex in the combination treatment of the present invention was replaced by the same doses of IgG antibodies(the same dose of IgG in histamine-immunoglobulin complex) from human gammaglobulin or IgG for intravenous administration made by same manufacturer for the histamine-immunoglobulin complex. The above results confirm that the therapeutic efficacy of the above combination of the present invention is not simply originated from the formation of immune complex made of allergen and allergen-specific antibodies as reported by Saint-Remy (Clin Exp Allergy 1994;24:1091-3). The above clinical trial examples also show that if the pharmaceutical composition of the present invention contains allergen-specific antibodies isolated from blood samples obtained from multiple normal controls together with histamine and allergen, the composition of the present invention can evidently result in the development of an advanced pharmaceutical composition and a new treatment method for allergic diseases that express both the synergistic effects of combining allergen, immunoglobulin, and histamine of the present invention and the therapeutic effect of immune complex made of allergen and allergen-specific antibodies reported by Saint-Remy simultaneously.

### INDUSTRIAL APPLICABILITY

If the pharmaceutical composition of the present invention is administered to the patients with allergic diseases, marked and clinically better improvement of allergic diseases can be obtained than the treatment with allergen-immunotherapy alone or histamine-immunoglobulin complex alone. Therefore, the allergic diseases can be significantly improved without side effects even in the patients with refractory allergic diseases who could not be sufficiently improved by treatment with standard drug therapy or allergen-immunotherapy if the pharmaceutical composition, its use for treating allergic diseases, or the treating method using the above compositions of the present invention was applied.

1-21. (canceled)

22. A pharmaceutical composition for treating an allergic disease, comprising as active ingredients:
histamine;
immunoglobulin; and
at least one allergen selected from the group consisting of house dust mites, pollen, animal dander, or fungus.

23. The pharmaceutical composition of claim 22, wherein the active ingredients are present in the form of dry powder.

24. The pharmaceutical composition of claim 22, wherein the allergic disease is selected from the group consisting of atopic dermatitis, allergic rhinitis, allergic conjunctivitis, urticaria, or allergic asthma.

25. The pharmaceutical composition of claim 22, wherein the histamine is present in a dose of from 0.05 to 2.5 micrograms.

26. The pharmaceutical composition of claim 22, wherein the immunoglobulin is present in a dose of from 0.05 to 50 milligrams.

27. The pharmaceutical composition of claim 22, wherein the allergen is present in a dose of from 1 to 1 000 micrograms.

28. A method for treating an allergic disease, comprising:
administering to a mammalian subject a pharmaceutical composition including a therapeutically effective amount of histamine, immunoglobulin, and at least one allergen selected from the group consisting of house dust mites, pollen, animal dander, or fungus.

29. The method of claim 28, wherein the pharmaceutical composition is in the form of dry powder, and
wherein the pharmaceutical composition is administered to the mammalian subject after the pharmaceutical composition is dissolved in buffer for injection.

30. The method of claim 28, wherein the allergic disease is selected from the group consisting of atopic dermatitis, allergic rhinitis, allergic conjunctivitis, urticaria, or allergic asthma.

31. The method of claim 28, wherein the histamine is present in a dose of from 0.05 to 2.5 micrograms.

32. The method of claim 28, wherein the immunoglobulin is present in a dose of from 0.05 to 50 milligrams.

33. The method of claim 28, wherein the allergen is present in a dose of from 1 to 1 000 micrograms.
